(19) United States
(12) Patent Application Publication
(10) Pub. No.: US 2021/0166789 A1
(43) Pub. Date: Jun. 3, 2021

(54) METHOD FOR IDENTIFYING GENE EXPRESSION SIGNATURES

(71) Applicant: SkylineDx B.V., Rotterdam (NL)

(72) Inventors: Martinus Hendrikus Van Vliet, Rotterdam (NL); Joske Ubels, Rotterdam (NL); Jeroen de Ridder, Utrecht (NL)

(21) Appl. No.: 16/500,379

(22) PCT Filed: Apr. 4, 2018
(86) PCT No.: PCT/NL2018/050207

§ 371 Date: Oct. 2, 2019

(30) Foreign Application Priority Data
Apr. 4, 2017 (EP) 17164855.3

(51) Int. Cl. G16B 40/30; G16H 10/60; G16H 10/40; G16H 50/20; G16H 50/70; G16H 70/60; G16H 70/20; G06N 20/00; G06N 7/00; G06F 16/28; G06F 16/2457

(52) U.S. Cl. CPC G16B 40/30; G16H 10/60; G16H 10/40; G16H 50/20; G16H 50/70; G06F 16/24578; G16H 70/20; G06N 20/00; G06N 7/005; G06F 16/285; G16H 70/60

(57) ABSTRACT

The disclosure relates to methods of identifying gene signatures that can be used in order to classify patients and predict responsiveness to therapy. In particular, the disclosure relates to TOPSPIN (Treatment Outcome Prediction using Similarity between PatIeNts)/GESTURE (Gene Expression-based Simulated Treatment Using similaRity between patiEnts), a new computational method to discover gene expression signatures capable of identifying a subgroup of patients more likely to benefit from a specific treatment as compared to another treatment.

### CROSS-REFERENCE TO RELATED APPLICATIONS

This application is a national phase entry under 35 U.S.C. § 371 of International Patent Application PCT/NL2018/050207, filed Apr. 4, 2018, designating the United States of America and published as International Patent Publication WO 2018/186740 A1 on Oct. 11, 2018, which claims the benefit under Article 8 of the Patent Cooperation Treaty to European Patent Application Serial No. 17164855.3, filed Apr. 4, 2017.

### TECHNICAL FIELD

The disclosure relates to methods of identifying gene signatures which can be used in order to classify patients and predict responsiveness to therapy. In particular, the disclosure relates to TOPSPIN (Treatment Outcome Prediction using Similarity between PatleNts)/GESTURE (Gene Expression-based Simulated Treatment Using similaRity between patiEnts), a new computational method to discover gene expression signatures capable of identifying a subgroup of patients more likely to benefit from a specific treatment as compared to another treatment.

### BACKGROUND

It is increasingly recognized that the successful treatment of cancer is hampered by genetic heterogeneity of the disease and a more personalized approach is needed. Differences in the genetic makeup between tumors can result in a different response to treatment (Burrell et al., 2013). Efforts to pair patients with therapies have been highly unsatisfactory, with 2-6% of cases assigned to a therapy (Prasad, 2016). As a result, despite the existence of a wide range of efficient cancer treatments available (Block et al., 2015), many therapies only benefit a minority of the patients that receive them, while they are associated with very serious side effects. For a few therapies targeting a specific mutation or protein, there are markers available like HER2 amplification for Herceptin in breast cancer (Molina et al., 2001), a specific EGFR mutation for erlotinib in lung cancer (Tsao et al., 2005) and presence of a KRAS mutation for cetuximab in colon cancer (Lièvre et al., 2006). Unfortunately, in absence of such markers it is often not clear which subset of the patients will respond well. Therefore, there is a great clinical need for tools to predict which patient will benefit most from which treatment.

### BRIEF SUMMARY

One aspect of the disclosure provides a machine-implemented method for identifying a gene signature for classifying a patient based on likelihood of response to a therapy of interest from a dataset comprising gene expression data and time until event data for a first group of subjects treated with the therapy and gene expression data and time until event data for a second group of subjects not treated with the therapy, the method comprising:
a) optionally, splitting the subjects into a validation group comprising subjects from both the first and second groups and a training group comprising subjects from both the first and second groups;
b) defining a ranked list of subjects from group 1, preferably of the training group from step a), that exhibit a greater treatment benefit over a set of genetically similar subjects from group 2, preferably of the training group from step a), as compared to the treatment benefit over a set of random subjects from group 2, preferably of the training group from step a),
wherein the treatment benefit is determined for functionally coherent gene sets
c) defining a classifier Q for each gene set i (Qi) by making a decision boundary defined in terms of an area (distance <γ) around z top-ranked subjects from step b), wherein z is at least 1, such that the hazard ratio for class 1 (all patients that fall into the area) is optimized, preferably wherein the decision boundary is such that the hazard ratio is associated with a p-value <0.05, wherein class 1 refers to the group of subjects from group 1 expected to respond to the therapy of interest;
d) determining the performance of classifier Q for each gene set using a gene expression dataset comprising a first group of subjects treated with the therapy and a second group of subjects not treated with the therapy and ranking classifiers Qi based on their hazard ratios, and
e) selecting the top k classifiers as the gene signature for classifying a patient. Preferably, wherein k is from 2 to 100, preferably k is from 4 to 10. More preferably, wherein k is from 2 to 400, for example, from 50 to 150. Preferably k is from 2 to 300.

Preferably, step b) of the method is performed by defining for each subject (i) from the first group of subjects the treatment benefit defined as

$${zPFS}_{i} = \frac{\frac{1}{n} {\sum }_{j ∈ O} ( {PFS}_{i} - {PFS}_{j} ) - μ ( {RPFS}_{i} )}{σ ( {RPFS}_{i} )}$$

wherein O is the set of the n most similar subjects based on distance from the second group of subjects (j), PFSi indicates the PFS of patient i and PFSj indicates the PFS of patient j, RPFS indicates a vector of ΔPFSi of patient i with differing random set of patients from the second group in O, μ indicates the mean, and σ indicates the standard deviation.

Preferably, step c) of the method) is performed by using the cosine correlation as distance measure. Preferably, step c) is performed by performing a grid search on all combinations of z and γ.

Preferably, step d) comprises determining the performance of Qi on the validation group of subjects.

One aspect of the disclosure provides a machine-implemented method for identifying a gene signature for classifying a patient based on likelihood of response to a therapy of interest from a dataset comprising gene expression data and time until event data for a first group of subjects treated with the therapy and gene expression data and time until event data for a second group of subjects not treated with the therapy, the method comprising identifying subjects from the first group that exhibit a greater treatment benefit over a set of genetically similar subjects from the second group as compared to the treatment benefit over a set of random subjects from group 2, wherein genetic similarity is determined based on expression of functionally coherent gene sets. Preferably the methods comprise identifying functionally coherent gene sets that are associated with the genetic similarity. Preferably, the methods comprise identifying a gene signature for classifying a patient based on likelihood of response to a therapy of interest, which is able to identify subjects from a first group treated with the therapy that exhibit a greater treatment benefit over a set of genetically similar subjects from a second group of subjects not treated with the therapy as compared to the treatment benefit over a set of random subjects from the second group. Preferably, the methods comprise one or more steps described herein. Preferably, the methods comprise step a) as described herein. Preferably, the methods comprise step b) as described herein. Preferably, the methods comprise step c) as described herein. Preferably, the methods comprise step d) as described herein. Preferably, the methods comprise step e) as described herein. Preferably, the methods comprise steps b)-e) as described herein.

One aspect of the disclosure provides a machine-implemented method for identifying a gene signature for classifying a patient based on likelihood of response to a therapy of interest, the method comprising identifying subjects from a first group treated with the therapy that exhibit a greater treatment benefit over a set of genetically similar subjects from a second group of subjects not treated with the therapy as compared to the treatment benefit over a set of random subjects from the second group, wherein genetic similarity is determined based on expression of functionally coherent gene sets. Preferably the methods comprise identifying functionally coherent gene sets that are associated with the genetic similarity. Preferably, the methods comprise identifying a gene signature for classifying a patient based on likelihood of response to a therapy of interest, which is able to identify subjects from a first group treated with the therapy that exhibit a greater treatment benefit over a set of genetically similar subjects from a second group of subjects not treated with the therapy as compared to the treatment benefit over a set of random subjects from the second group.

Preferably, the methods comprise one or more steps described herein. Preferably, the methods comprise step a) as described herein. Preferably, the methods comprise step b) as described herein. Preferably, the methods comprise step c) as described herein. Preferably, the methods comprise step d) as described herein. Preferably, the methods comprise step e) as described herein. Preferably, the methods comprise steps b)-e) as described herein.

Preferably, the methods described herein comprise:
f) repeating step a) by splitting the subjects into a new validation group comprising subjects from both the first and second groups and a new training group comprising subjects from both the first and second groups;
g) repeating step b);
h) repeating step c); and
i) determining the performance of classifier Q for each gene set using a gene expression dataset comprising a first group of subjects treated with the therapy and a second group of subjects not treated with the therapy and ranking classifiers Qi based on mean hazard ratios from step h).

Preferably, second group of subjects is treated with an alternative therapy.

Preferably, the functionally coherent gene sets are obtained from the Gene Ontology (GO) categories.

Preferably, the time until event refers to Progression Free Survival (PFS). Preferably, the patient is classified as Class 1 or Class 0. In some embodiments, each classifier of the gene signature classifies as Class 1 or Class 0. The patient is classified by a threshold of the number of classifiers that indicate a particular class.

### BRIEF DESCRIPTION OF THE DRAWINGS

FIG. 1. A. The input data for building gene signatures; a patient by gene expression matrix is supplemented with prognostic class labels and treatment information B. An example of a prognostic classification, where there is a significant survival difference between the two classes. C. An example of a predictive classification, where there is a significant survival difference between the two treatments within one class. D. Kaplan Meier of the Multiple Myeloma dataset.

FIG. 2. Flow of the TOPSPIN algorithm. First, zPFS is calculated on fold A to rank possible prototypes (Step 1). Then z (number of prototypes) and γ (radius around the prototypes) parameters are optimized in fold B. The optimal combination is chosen, i.e., the one resulting in the lowest HR and conforming to minimum class size and p-value constraints (Step 2). The performance of this combination is measured on fold C and sets are ranked by their performance (Step 3). For each patient the score for the top 8 gene sets, based on mean HR, is calculated. The final class labels are obtained by thresholding this score. The final classifier is validated on fold D (Step 4).

FIG. 3. A. The HRs found for the top 8 GO sets over ten repeats, ranked on mean HR. B. HR and group size in training set with different thresholds t. C. Kaplan Meier of the classification found in the training set (Fold ABC). D. Kaplan Meier of the independent validation (Fold D1).

FIG. 4. Gene network built with genes used in the GO classifiers. Red nodes are used in the classifier for fold D1, purple nodes in the classifier for fold D2, blue nodes in the classifier for fold D3. Edges indicate either co-expression (purple edge), a physical interaction (red edge), co-localization (blue edge) or a shared protein domain (gold edge).

FIG. 5. Performance of adding GO gene set to classifier in 1 repeat. Here the optimal number of sets is 8: this was the median number of gene sets selected over 5 repeats.

FIG. 6. A. Kaplan Meier of training performance for fold D2. B. Kaplan Meier of test performance for fold D2. C. Kaplan Meier of training performance for fold D3. D. Kaplan Meier of test performance for fold D3.

FIG. 7. a. Example of the Kaplan Meier curve for a prognostic classifier. b. Example of the Kaplan Meier curve for a predictive classifier. c. Division of dataset into training and test sets. D1, D2 and D3 are all used once to validate the classifier trained on the remaining two thirds of data. d. Flow of the GESTURE algorithm. In step 1 the prototypes with a longer than expected survival difference are identified on fold A. In step 2 the number of prototypes and corresponding decision boundary used in the classifier are optimized on fold B. In step 3 the performance of the classifier on fold C across all repeats is used to select the combination of gene sets to be used in the final classifier. In step 4 a classifier for these gene sets is defined on all training data. This classifier will be validated on the fold D not included in the training data.

FIG. 8 a. Kaplan Meier of the entire bortezomib dataset, showing a HR of 0.74 (p=0.0029) between the treatment arms. b. Kaplan Meier of the combined classifications into a ‘benefit’ and ‘no benefit’ class of D1, D2 and D3. A HR of 0.50 (p=0.0012) is found between the treatment arms in the ‘benefit’ class and a HR of 0.78 (p=0.03) in the ‘no benefit’ class. c. The HR found in the ‘benefit’ class (y-axis) when different operating points (x-axis) are used, compared with known predictive and prognostic markers. The gray dotted line indicated the HR found in the entire dataset, without classification. d. Relationships between the 31 genes in common between the D1, D2 and D3 classifiers. Node size corresponds to how much more a gene was observed in the selected gene sets than expected. Green nodes indicate that the gene is associated with a p-value <0.05. Relationships are inferred from literature with the GeneMANIA40 algorithm. A purple edge indicates the genes are co-expressed, a green edge indicates a genetic interaction, a red edge a physical interaction, an orange edge a shared protein domain, a dark blue edge indicates co-localization and a light blue edge shows that both genes are annotated to the same pathway.

FIG. 9. Number of classifiers that predict benefit, measured per patient over three classifier. Red dots are the overlap found between the three STL classifiers, compared to 10 000 randomly generated classifications with the same percentages class ‘benefit’ (boxplot).

FIG. 10. Kaplan Meier of the classification of the bortezomib dataset using random gene sets.

### DETAILED DESCRIPTION

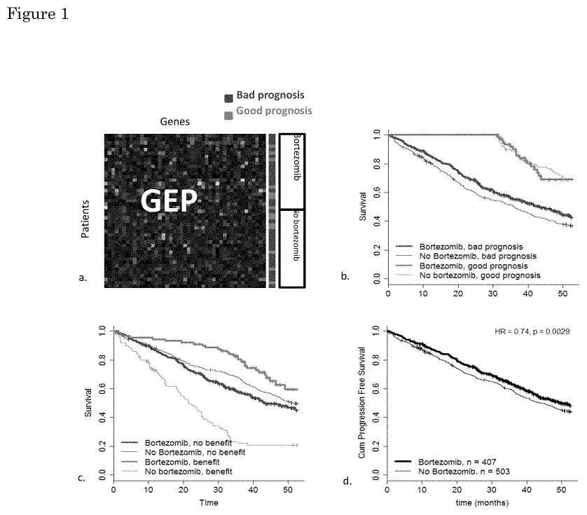

Current efforts to address patient prognosis are often aimed at discovering molecular cancer subtypes and determine a gene expression signature that differentiates between them (Davis and Staudt, 2002). In case patient follow-up survival data is available, supervised learning approaches can be used to directly obtain a prognostic signature. Thus to obtain a prognostic signature, gene expression data and survival information (FIG. 1a) are needed, and a classifier is trained that should result in a significant survival difference between the two classes (FIG. 1b). One of the first successful demonstrations of such an approach yielded a 70-gene signature that can distinguish breast cancer patients with a poor prognosis that may benefit from adjuvant therapy from those with a good prognosis that would not benefit from adjuvant therapy (Van 't Veer et al., 2002). Since then, many prognostic classifiers and signatures have been published for a variety of cancers (Santos et al., 2015). For example, EP2546357 describes a gene signature for determining the prognosis of a patient diagnosed with multiple myeloma. Based on the gene signature, a patient is classified in a high risk or low risk category indicative of overall survival rates.

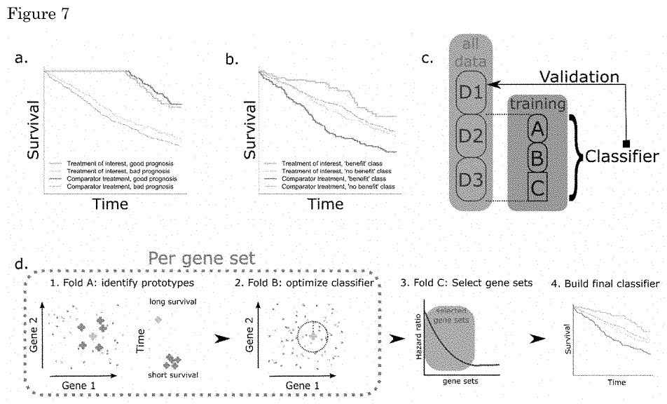

By definition, prognostic classifiers predict survival irrespective of which treatment the patient receives and are thus unsuited to predict which patients benefit from a particular treatment of interest. The efficacy of novel therapies is evaluated in randomized phase III clinical trials, typically comparing the novel treatment to a current standard-of-care. In such trials, patients are randomly assigned to a particular treatment regime, mitigating confounding factors. Constructing classifiers that can achieve true treatment benefit prediction thus poses a unique challenge, as it is impossible to know how a patient would have responded to the alternative treatment. As a result, class labels based on which can be used to train a classifier are not available and existing classification schemes are not applicable. When the clinical trial includes the measurement of gene expression data, a unique opportunity is created to infer predictive classifiers, i.e., classifiers able to predict which patients benefit from a particular treatment of interest (FIGS. 1c and 7b).

Treatment benefit is commonly measured by the Hazard Ratio (HR), which describes a patient's hazard to experience an event, for example, death or progression of disease, relative to another set of patients who received a different treatment. Some recently published predictive classifiers, have only shown to find a difference in response or survival between two groups of patients who all received the same treatment (Bhutani, M., et al. Investigation of a gene signature to predict response to immunomodulatory derivatives for patients with multiple myeloma: an exploratory, retrospective study using microarray datasets from prospective clinical trials. The Lancet Haematology, 4, e443-e451. doi:10.1016/S2352-3026(17)30143-6 (2017).

Vangsted et al., Drug response prediction in high-risk multiple myeloma. Gene https://doi.org/10.1016/j.gene.2017.10.071 (2017); Ting, K. R., et al. Novel panel of protein biomarkers to predict response to bortezomib-containing induction regimens in multiple myeloma patients. BBA Clinical, 8, 28-34. (2017)). These signatures are not constructed to be predictive, since they do not necessarily provide a treatment decision; the prognosis may well be the same in every treatment group. To be truly predictive, a subgroup with a difference in survival between two treatment arms needs to be identified.

A successful predictive classifier ideally results in a survival curve similar to the one depicted in FIGS. 1c and 7b, that is, based on the gene expression data a class of patients can be distinguished for which the treatment of interest has a significant survival benefit. Inferring such a classifier is challenging because it is unknown what a patient's survival would have been if the patient would have received the other treatment. Moreover, patients in this class may not necessarily have a good prognosis overall; their prognosis is merely better than it would have been if they were given the other treatment. As a result, such classifiers cannot be obtained with a standard binary or multiclass problem formulation. For instance, labelling patients receiving the treatment of interest with good prognosis as positive and the rest as negative fails to yield good classifier performances.

The present disclosure provides a new type of classifier, TOPSPIN (Treatment Outcome Prediction using Similarity between PatIeNts), also known as GESTURE (Gene Expression-based Simulated Treatment Using similaRity between patiEnts), that derives a classifier that is able to identify a subgroup of patients more likely to benefit from a specific treatment as compared to another treatment. The methods disclosed herein are based in part on the idea that a patient's treatment benefit can be determined by comparing survival of a patient (or another time to event) to the survival of a set of genetically similar patients that received a different treatment. In the case where genetics determines prognosis, the comparison to genetically similar patients is introduced to try to mimic the outcome of the patient if they had received the alternative treatment. The concept of Simulated Treatment Learning (STL) has been implemented, in the algorithm GESTURE, which makes it possible to derive a gene expression signature that is able to distinguish a subset of patients with improved treatment outcome from the treatment of interest, but not from the comparator treatment.

Genetic similarity can be defined in many different ways. In the present examples, genetic similarity is based on the gene expression data at hand, and genetic similarity is defined in terms of the (preferably, cosine) similarity across functionally related genes. Patients with large treatment benefit serve as prototypes: newly diagnosed patients similar to this prototype can then be classified as benefitting from treatment. Thus, gene signatures that identify prototypes can also be used to classify patients.

Unlike many of the prior art methods, the present methods do not make any assumptions a priori regarding which biological features (e.g., biological pathways, chromosomal alterations, etc.) may result in differences in treatment. In fact, the present methods may be used to determine whether there are patient populations that demonstrate a large treatment benefit for a particular therapy in contrast to therapies that demonstrate small treatment benefits, but more consistently over the entire patient population as a whole.

The examples herein describe the application of the TOPSPIN method in a multiple myeloma dataset, where patients enrolled in a phase III clinical trial either received the proteasome inhibitor bortezomib or not. Gene sets were identified that can identify a subset of patients in which a significant hazard ratio between the bortezomib group and the group who received conventional therapy is found. The classifier trained with these gene sets validates on independent test data. The classifier identified by the TOPSPIN method outperforms classifiers trained using a nearest mean classifier, random forest and support vector machine.

The disclosure provides machine-implemented methods for identifying a gene signature for classifying a patient based on likelihood of response to a therapy of interest from a dataset comprising gene expression data and time until event data for a first group of subjects treated with the therapy and gene expression data and time until event data for a second group of subjects not treated with the therapy. The methods allow a patient to be classified. The patient may be classified as Class 1 or Class 0. Class 0 refers to patients that do not benefit, or are not expected to benefit, from the therapy of interest as compared to not receiving the treatment. When the subjects of group 2 in the methods disclosed herein receive no treatment or placebo treatment, then Class 0 patients represent those patients that respond no better to the therapy of interest than placebo. Such patients are not expected to receive any benefit from the therapy of interest. When the subjects of group 2 in the methods disclosed herein receive an alternative treatment (e.g., the standard of care treatment) then Class 0 patients represent those patients that respond no better to the therapy of interest than the alternative treatment. Such patients are expected to benefit from the therapy of interest, but the therapy does not exhibit an improvement over the alternative treatment.

Class 1 refers to patients that benefit, or are expected to benefit, from the therapy of interest. When the subjects of group 2 in the methods disclosed herein receive no treatment or placebo treatment, then Class 1 patients represent those patients that respond better to the therapy of interest than placebo. When the subjects of group 2 in the methods disclosed herein receive an alternative treatment (e.g., the standard of care treatment) then Class 1 patients represent those patients that respond better to the therapy of interest than the alternative treatment.

A skilled person is able to determine when a greater treatment benefit (or difference in time to event) is significant. Preferably, the significance is p>0.05. In some embodiments, the time to event is more than 10%, more than 20%, or more than 50% longer for the greater treatment benefit.

The disclosure provides machine-implemented methods for identifying a gene signature for classifying a patient based on likelihood of response to a therapy of interest. Preferably, the methods use a dataset comprising gene expression data and time until event data for a first group of subjects treated with the therapy and gene expression data and time until event data for a second group of subjects not treated with the therapy. The methods comprise identifying “prototypes”, or rather subjects from the first group that exhibit a greater treatment benefit over a set of genetically similar subjects from the second group as compared to the treatment benefit over a set of random subjects from group 2. Gene sets that are able to successfully identify prototypes may be used as a gene signature for classifying a patient based on likelihood of response to a therapy of interest.

Preferably, genetic similarity is determined by defining a classifier Q for each gene set i (Qi) by making a decision boundary defined in terms of an area (distance <γ) around subjects identified from a first group treated with the therapy that exhibit a greater treatment benefit over a set of genetically similar subjects from a second group of subjects not treated with the therapy as compared to the treatment benefit over a set of random subjects from the second group, such that the hazard ratio for class 1 (all patients that fall into the area) is optimized, preferably wherein the decision boundary is such that the hazard ratio is associated with a p-value <0.05, wherein class 1 refers to the group of subjects from group 1 expected to respond to the therapy of interest.

Preferably, the methods comprise step b) defining a ranked list of subjects from group 1 that exhibit a greater treatment benefit over a set of genetically similar subjects from group 2 as compared to the treatment benefit over a set of random subjects from group 2, wherein the treatment benefit is determined for functionally coherent gene sets. Subjects that exhibit a greater treatment benefit over a set of genetically similar subjects from group 2 as compared to the treatment benefit over a set of random subjects from group 2 of the training group are referred to herein as “prototypes”. The term greater treatment benefit may be defined according to the time to event. For example, if the event is PFS, then a greater treatment benefit refers to a longer progression free survival, if the event is OS, then a greater treatment benefit refers to a longer overall survival.

Functionally coherent gene sets are known to a skilled person. Preferably, the GO terms offered by the Gene Ontology Consortium and found on the world wide web at geneontology.org can be used, or rather the gene sets are GO gene sets. Gene Ontology (GO) categories show the relationships between genes and the keywords assigned for each gene and it is applicable to bioinformatics. Generally, GO terms are classified into three categories reflecting the biological roles of genes: i) molecular function, ii) biological process and iii) cellular component. Hierarchically controlled vocabularies are established for each category. The three categories are not exclusive but are descriptive of a gene.

It is preferred that the methods include as many functionally coherent human gene sets as possible as this will improve the ability to identify relevant gene sets. As is clear to a skilled person, some gene sets will exhibit little if any variability between subjects either from the same group or between groups (e.g., housekeeping genes). Such gene sets are preferably not included in the methods as they will not aid in identifying prototypes nor will they distinguish differences in response. Similarly, some genes/probesets within a functionally coherent gene set will exhibit little if any variability. Such genes/probesets are preferably not included in the methods, which results in a reduction of the genes/probesets need to be tested in a functionally coherent human gene sets.

A preferred method for analysing variability is described in the examples. Samples were processed on a microarray and gene expression was normalized. The resulting data was scaled to mean 0 and variance 1. By way of example only, a functionally coherent human gene set with 10 genes/probesets, may exhibit a variance of >1 for only 6 of the probesets. For such a case, the methods disclosed herein would test the gene set but only in regards to those 6 probesets. While the 4 probesets having a variance of <1 may be included, a skilled person would recognize that they will not provide any useful information to distinguish subjects.

In the present examples, the number of subjects in the set of genetically similar subjects from group 2 of the training group was 10. However, this number can be smaller or larger and may depend on the number of subjects in the dataset. The number of random subjects from group 2 of the training group may be equal to the number of subjects in the set of genetically similar subjects from group 2 of the training group. It may also be smaller or larger, and in some embodiments it may include all of the subjects from group 2 of the training group.

The methods disclosed herein may comprise step a) splitting the subjects into a validation group comprising subjects from both the first and second groups and a training group comprising subjects from both the first and second groups. In such a case the subjects used in step b) may be from the training group.

Preferably, the methods further comprise step c) defining a classifier Q for each gene set i (Qi) by making a decision boundary defined in terms of an area (distance <γ) around z top-ranked subjects from step b), wherein z is at least 1, such that the hazard ratio for class 1 (all patients that fall into the area) is optimized, preferably wherein the decision boundary is such that the hazard ratio is associated with a p-value <0.05, wherein class 1 refers to the group of subjects from group 1 expected to respond to the therapy of interest.

Preferably, step c) is performed by performing a grid search on all combinations of z and γ. As known to a skilled person, “grid searching” (or parameter sweep) is a form of hyperparameter optimization used in the context of machine learning. Essentially, it is an exhaustive searching through a specific subset of the hyperparameter space of a learning algorithm (e.g., Chin-Wei Hsu, Chih-Chung Chang and Chih-Jen Lin 2010 A practical guide to support vector classification. Technical Report, National Taiwan University). In the present methods, the searched subsets include all combinations of z and γ.

Preferably, the methods further comprise step d) determining the performance of classifier Q for each gene set using a gene expression dataset comprising a first group of subjects treated with the therapy and a second group of subjects not treated with the therapy and ranking classifiers Qi based on their hazard ratios.

Preferably, the methods further comprise step e) selecting the top k classifiers (ranking determined in step d)) as the gene signature for classifying a patient, wherein k is from 2 to 100, preferably k is from 4 to 10. In some embodiments, the top k classifiers may be from 2 to 300, although generally k will be around 100.

Preferably, the methods comprise steps b)-e).

In one embodiment, the method steps are repeated by:
f) providing a new set of group 1 patients and a new set of group 2 patients. Preferably, this is performed by splitting the subjects into a new validation group comprising subjects from both the first and second groups and a new training group comprising subjects from both the first and second groups. While this step (re-splitting) can be performed on the initial dataset of patients, as an alternative, the data from subjects from an entirely new dataset can be used. The method further comprises:
g) repeating step b);
h) repeating step c); and
i) determining the performance of classifier Q for each gene set using a gene expression dataset comprising a first group of subjects treated with the therapy and a second group of subjects not treated with the therapy and ranking classifiers Qi based on mean hazard ratios from step h). The top k classifiers are then selected for the gene signature.

The methods provide gene signatures that can be used to classify a patient. In particular, the patient may be classified as Class 1 or Class 0, as described further herein. In one embodiment, the disclosure provides methods for classifying a patient comprising:
a) determining the gene expression levels of a gene signature comprising k classifiers obtained as disclosed herein; and
b) classifying the patient as Class 1 if at least t of the k classifiers indicates Class 1, wherein t (threshold) is defined as from 1-k.

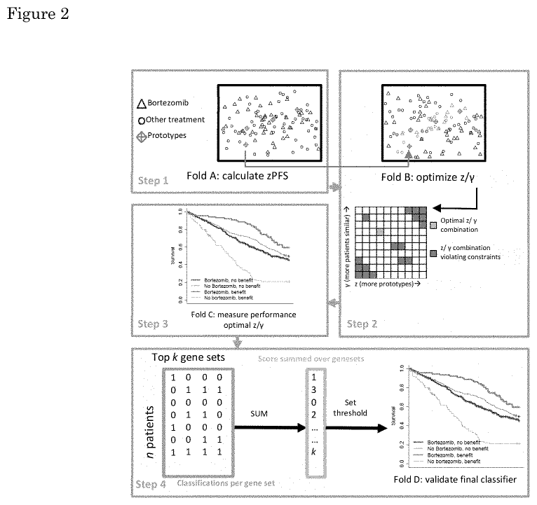

In the present examples, a gene signature having 8 classifiers was identified and a t of 3 is used (see, e.g., FIG. 2, step 4). However, as is clear to a skilled person, the threshold will vary depending on, e.g., the desired sensitivity and/or specificity to be achieved.

The dataset comprises gene expression data, preferably nucleic acid expression data. There are many published sources of gene expression data, e.g., those obtained from published clinical trials. Gene expression data may also be determined as part of the methods. Determining the level of expression includes the expression of nucleic acid, preferably mRNA, or the expression of protein. In some embodiments, nucleic acid or protein is purified from the sample and expression is measured by nucleic acid or protein expression analysis. It is clear that the choice of sample will depend on the therapy of interest and the conditions it is being used to treat. For example, when investigating therapies for treating multiple myeloma, preferably, the sample comprises plasma cells. Although a preferred source of plasma cells is a bone marrow sample, other plasma cell containing samples, such as, e.g., blood, may also be used.

Expression data preferably refers to the level of nucleic acid corresponding to the probes used for detection or the corresponding genes they refer to. Suitable probes include those commercially available on DNA microarrays, such as Affymetix™ chips. It is well within the purview of a skilled person to develop additional probes for determining expression. The level of nucleic acid expression may be determined by any method known in the art including RT-PCR, quantitative PCR, Northern blotting, gene sequencing, in particular, RNA sequencing, and gene expression profiling techniques. Preferably, the level of nucleic acid using a microarray.

Preferably, the nucleic acid is RNA, such as mRNA or pre-mRNA. As is understood by a skilled person, the level of RNA expression determined may be detected directly or it may be determined indirectly, for example, by first generating cDNA and/or by amplifying the RNA/cDNA. The level of expression need not be an absolute value but rather a normalized expression value or a relative value.

Preferably, the level of expression refers to a “normalized” level of expression. Normalization is particularly useful when expression is determined based on microarray data. Normalization allows for correction for variation within microarrays and across samples so that data from different chips can be simultaneously analyzed. The robust multi-array analysis (RMA) algorithm may be used to pre-process probe set data into gene expression levels for all samples. (Irizarry R A, et al., Biostatistics (2003) and Irizarry R A, et al., Nucleic Acids Res. (2003)). In addition, Affymetrix's default preprocessing algorithm (MAS 5.0), may also be employed. Additional methods of normalizing expression data are described in US20060136145.

The disclosed methods may be used to identify a gene signature for classifying patients based on their likelihood to respond to any therapy of interest. The term “likelihood to respond” refers to the probability of an event and, for example, may refer to the likelihood that patient survival will increase as a result of the therapy of interest. As is clear to a skilled person, the term likelihood to respond refers to a probability and not that 100% of all patients that are predicted to respond to a treatment may actually respond.

Ideally, the second group of subjects in the methods are not treated or are only given a placebo. The disclosed methods would thus identify gene signatures that predict responsiveness of a therapy over no treatment. As is well-known to a skilled person, for many diseases it is not possible to have a placebo arm. Rather, a new treatment may be compared to the standard of care. In such cases, the disclosed methods are useful for identifying gene signatures that can predict an increase in responsiveness to the new therapy over the standard of care. In some embodiments, the methods are for classifying a patient, in particular, for classifying as benefiting from a therapy of interest as compared to no treatment or an alternative treatment.

The dataset comprises data on time until event. Response to treatment can be measured by any number of time to events/endpoints including time-to-disease-progression (TTP), Overall Survival (OS), or Progression Free Survival (PFS). Preferably, the time to event is PFS. In addition, the time to event can also include the time until a tumor reaches a particular size or the time until a particular symptom appears. An individual classified as a likely responder to a therapy (Class 1) is predicted to respond better when administered the therapy over the alternative therapy.

Preferably, the therapy is a cancer therapy, in particular, a therapy for treating Multiple Myeloma (MM). Preferably, the therapy is an Immunomodulatory drug therapy (IMiDs), such as thalidomide and lenalidomide. Preferably, the therapy is a proteasome inhibitor therapy such as bortezomib.

One of the advantages of applying the methods disclosed herein to predict response is that it allows for optimizing a treatment regime. Individuals that are predicted to respond to a particular treatment may be subsequently administered such treatment. Conversely, individuals predicted not to respond to a particular treatment may be administered with an alternative treatment. This can result in a decrease in unnecessary treatments.

### Definitions

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, the additional component(s) not altering the unique characteristic of the disclosure.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

All patent and literature references cited in the present specification are hereby incorporated by reference in their entirety.

This disclosure is further explained in the following examples. These examples do not limit the scope of the disclosure, but merely serve to clarify the disclosure.

### EXAMPLES

The examples describe the application of the TOPSPIN classifier on Multiple myeloma (MM), which is a clonal B-cell malignancy that is characterized by abnormal proliferation of plasma cells in both the bone marrow and the extramedullary sites. Median survival is 5 years (Howlader, 2016). In the last two decades many novel therapies have been introduced for MM, resulting in an improved survival (Kumar et al., 2008; Munshi and Anderson, 2013). However, response rates remain low and there is no clear indication what determines treatment response. This is complicated by the fact that MM is very heterogeneous, both between and within patients (Lohr et al, 2014; Keats et al., 2012). Especially in MM, predictive signatures could be of great benefit.

The examples describe the identification of a predictive classifier for the proteasome inhibitor bortezomib. In MM it was shown that patients with the chromosomal aberration del17p, known to be prognostic, benefitted more from the proteasome inhibitor bortezomib than patients without del17p (Neben et al., 2012). Problematically, this prognostic marker is only present in a small subset of the patients. Another, more commonly found, chromosomal aberration is the deletion of 1p22. Expression levels of a tumor suppressor located on this chromosome, RPL5, were found to correlate with bortezomib response (Hofman et al., 2015). Both these aberrations have been found to be recurrently present in MM plasma cells, and were later found to be prognostic and predictive. Instead of testing known markers, a new method was applied to directly discover gene signatures that are predictive from gene expression data.

### Example 1

Methods

Data and Processing

We pooled gene expression and survival data from three phase III trials: Total Therapy 2 (TT2, GSE2658, Barlogie et al., 2006), Total Therapy 3 (TT3, GSE2658, Barlogie et al., 2007) and HOVON-65/GMMG-HD4 (H65, GSE18784, Sonneveld et al., 2013). The TT2 dataset included 345 newly diagnosed multiple myeloma (NDMM) samples, treated either with thalidomide and melphalan (n=173) or melphalan alone (n=172). The TT3 dataset included 238 NDMM samples treated with bortezomib, thalidomide and dexamethasone (VTD). The H65 dataset included 327 NDMM samples, treated either with vincristine, doxorubicin and dexamethasone (VAD, n=169) or bortezomib, doxorubicin and dexamethasone (PAD, n=158). In the analyses of the pooled data two treatment arms were considered: a bortezomib arm, which comprises the PAD arm from H65 and TT3, and a non-bortezomib arm, which comprises the VAD arm from H65 and TT2.

All samples were profiled with the Affymetrix Human Genome U133 plus 2.0 array. Gene expression was MASS and log 2 normalized. Batch effects resulting from pooling different datasets were corrected with ComBat (Johnson et al., 2007). Data was scaled to mean 0 and variance 1. Probe sets with a variance of <1 before scaling were discarded.

The data was split in fold D1 (304 samples), fold D2 (303 samples) and fold D3 (303 samples), stratifying for treatment arm and survival. To prevent experimenter bias, fold D1 is not used at any point in the development and training of the algorithm and is solely used to validate the developed classifier. Fold D2 and fold D3 are combined to serve as training data. After the TOPSPIN classifier is successfully validated on fold D1, fold D2 and fold D3 are rotated to serve as additional validation folds to assess robustness.

Endpoint and Survival Analyses

Progression Free Survival (PFS) was used as endpoint, as this was the most direct readout of treatment related survival and therefore considered to be more relevant compared to overall survival. PFS times in the TT2 and H65 datasets were truncated to 52.53 months, corresponding to the longest follow-up time in the TT3 dataset.

Survival analyses were done using the Cox Proportional Hazards model (survival package, version 2.38.4, Therneau, 2015), stratified for dataset of origin. This means the β coefficient in the model was estimated separately for the TT2/TT3 dataset and the H65 dataset. This is necessary to correct for significant survival difference found between these datasets. Hazard Ratios (HR) and associated 2-sided p-values were calculated. P-values below 0.05 were considered statistically significant. All calculations were performed in R version 3.1.2.

Gene Ontology Gene Sets

We tested all Gene Ontology (GO) categories, as defined by the R Bioconductor package hgu133plus2.db (Carlson, 2016), with more than one probe set associated to them. This resulted in 10,581 gene sets. To test whether the biological information, contained in the GO annotation, aids the performance of the algorithm, 10,581 random gene sets matching the size of the actual selected GO categories were also tested.

Algorithm

TOPSPIN aims to predict if a patient benefits (class 1) or does not benefit (class 0) from a certain treatment of interest based on the gene expression profile of the patient. The term “does not benefit” refers to not benefiting over the alternative treatment. In order to train this classifier, a ranked list of prototype patients (Step 1) is defined that exhibit a better than expected prognosis compared to a set of genetically similar patients that received the opposite treatment. In Step 2, a decision boundary around a selection of prototype patients is determined, such that the HR for class 1 is optimized. Because it is a priori unknown based on which genes patient similarity should be defined, Step 1 and 2 are performed for a large number of functionally coherent gene sets obtained from the GO, yielding one classifier per gene set. In Step 3, a selection of well-performing classifiers is made, which are combined to obtain the final classifier in Step 4. These steps are visualized in FIG. 2.

In order to prevent overtraining, the training set is split into three equal training folds (A, B & C) and perform Steps 1-3 each on one of these separate training folds. The final classifier is based on all three training folds together.

Step 1—Prototype Ranking on Fold A

For each patient receiving the treatment of interest, the treatment benefit is defined as

$$Δ {PFS}_{i} = \frac{1}{n} {\sum }_{j ∈ O} ( {PFS}_{i} - {PFS}_{j} ) ,$$

where O is the set of the n most similar patients (based on cosine distance) that did not receive the treatment of interest. ΔPFS is only calculated for neighbour pairs where it is clear which patient experienced an event first; if both are censored, ΔPFS is not computed. To correct for the fact that a patient with a long survival time will, on average, have a large ΔPFS irrespective of its relative treatment benefit compared to genetically similar patients, the z-normalized zPFS score is defined as:

$${zPFS}_{i} = \frac{Δ {PFS}_{i} - μ ( {RPFS}_{i} )}{σ ( {RPFS}_{i} )} ,$$

where RPFS is a distribution of 1000 random ΔPFS scores, obtained by calculating ΔPFS for randomly chosen sets O, i.e., determining treatment benefit with respect to random patients instead of genetically similar patients. Based on the zPFS score, all patients in fold A that were given the treatment of interest can be ranked.

Step 2—Classifier Definition on Fold B

Classifier Q is defined by a subset of z top-ranked prototypes along with a decision boundary defined in terms of the cosine distance γ around a prototype. A patient is classified as class 1 when it lies within γ of any of the top z prototypes. The optimal values for z and γ are those resulting in the lowest Hazard Ratio (HR) in class 1 (the patient group in which the treatment of interest should have a better survival). Additionally, z and γ are constrained, such that class 1 comprises at least 20% of the dataset and the HR is associated with a p-value <0.05. The search grid for parameter z was 1 to 40 in steps of 1. The search grid for parameter γ was made dependent on the local density of the neighbours, and consisted of the sorted list of cosine distances between the prototype and its neighbours.

Step 3—Ranking Classifiers on Fold C

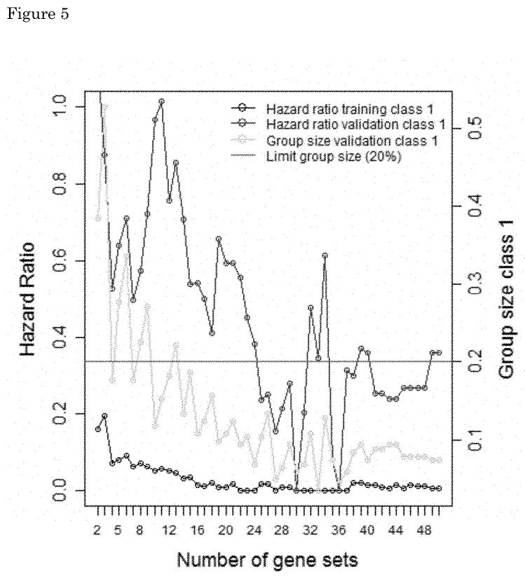

One classifier Qi, with i={1 . . . 10,581}, is trained for each GO gene set. Subsequently, the performance of all classifiers, defined by the HR obtained for class 1, is estimated based on fold C. To get a robust estimate of performance, this procedure was repeated 5 times, each with a new split into three folds. Moreover, for each repeat, fold A and B are swapped, so that for each gene set 10 repeats are essentially obtained. Classifiers Qi are ranked on their mean HR across the repeats. For the final classifier, the top k classifiers are selected using the classifier settings for z and γ from the repeat with the lowest HR. To empirically determine a good value for k, the classifiers within each repeat were ranked on their performance on fold A+B and the combined performance of the top k was tested on fold C, using values for k from 2 to 100. The k that resulted in the lowest HR on fold C was chosen. The median value of k across all repeats was 8 (FIG. 5), which was subsequently used for the final classifier. Note that steps 1-3 do not involve any of the 304 patients in the test set (fold D1).

Step 4—Combining Classifiers

Based on the k selected classifiers, all 606 patients in the training set (previously split in folds A, B and C) are reclassified. This gives each patient a score between 0 and k when k classifiers are used. To translate this score to a binary classification a threshold t is set, where a patient is classified as benefiting from treatment (class 1) when they are classified as such by t or more classifiers. This threshold is chosen so that the HR in class 1 is minimal, while containing at least 20% of the patients.

Comparison to Other Classifiers

We compared the performance of TOPSPIN to that of other classifiers in two ways. First, labels were defined by classifying the 25% longest surviving bortezomib patients and the 25% shortest surviving non-bortezomib patients as class 1. A classifier was trained using folds A-C to predict these labels, using the HR in validation fold D1 as performance measure. For the nearest mean classifier, a double-loop cross-validation was used to optimize the number of genes (ranked based on t-score), using balanced accuracy as the performance measure.

A random forest classifier (R package randomForest, version 4.6.12, Liaw and Weiner, 2002) and a support vector machine (R package e1071, version 1.6.7, Meyer et al., 2015) were also trained. For both these classifiers, the number of genes was optimized in cross validation. For the support vector machine values for C from 1 to 1000 were tested, in steps of 1. The gamma used is 1/P, where P is the number of input variables.

The labels found by TOPSPIN on the train set, when building a classifier to predict fold D1, were also used as training labels for these classifiers.

### Results

State-of-the-Art Classifiers Fail to Provide Performance

First, the aim is to determine whether it is possible to find a treatment-specific classifier by defining appropriate binary labels, and using regular binary classifiers such as a linear classifier or random forest. Such classifiers should improve over the HR of 0.74 (p=0.0029), which is the HR observed for the entire dataset (FIG. 1d). To this end, class 1 was first defined, the class for which patients should benefit more from bortezomib, as the 25% longest surviving bortezomib patients and the 25% shortest surviving non-bortezomib patients. A nearest mean classifier was trained, a random forest and a support vector machine to distinguish these patients from the rest of the population. The performance of these classifiers is summarized in Table 1. While their training performance is reasonable, all of these classifiers do not improve upon the baseline HR (FIG. 1d) and therefore failed to validate on new and unseen data. Similar poor performance was found when other percentages were used to define class 1 (data not shown). This demonstrates that, it is impossible to straightforwardly determine an appropriate class 1 for training from the survival data.

In an orthogonal effort, 1000 random labelings of the patients were generated and those that resulted in the top 10 lowest significant (p<0.05) HRs in class 1 were used to train a classifier. This approach also failed, since none of the trained classifiers resulted in a significant HR in class 1 when validated on fold D1. For each classifier the results for the labeling that resulted in the best training performance, as measured by balanced accuracy, are reported in Table 2. Notably, not only do none of them achieve a significant HR in the test set, these results demonstrate that a higher accuracy in predicting the class labels does not result in a better HR. This indicates that a major part of developing a predictive classifier is finding the appropriate labels. An important feature of TOPSPIN is that it automatically finds these labels, rather than using predefined ones.

TOPSPIN Validates on Independent Test Set

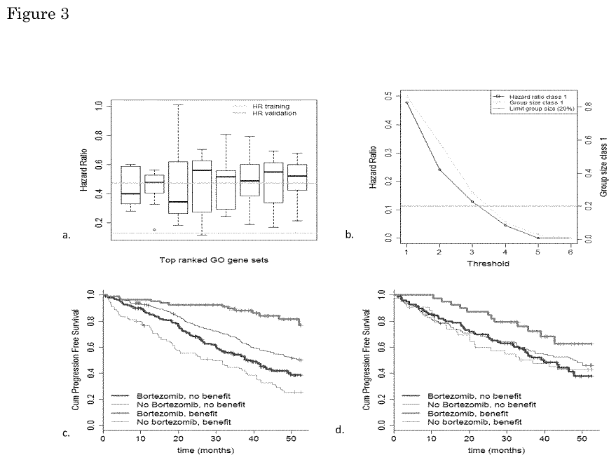

TOPSPIN was applied to the training dataset, with bortezomib being the treatment of interest. The training dataset, comprising folds D2 and D3, was used as input. Gene sets were defined by GO annotation. The performance of the top 8 classifiers, each associated to on GO gene set, across 10 repeats is shown in FIG. 3a. All top gene sets have a wide range in their performance in fold C, which seems to indicate that the algorithm is greatly influenced by which part of the data is used in training. This means it is vital to have multiple repeats to accurately estimate the performance of a gene set.

Combining the classifiers associated to the top gene sets yields improved performance, as can be observed from the green line, which shows the training-HR after combining these. This HR was obtained with a threshold t=3, i.e., a patient is classified as class 1 if three or more individual classifiers indicate class 1. When a higher threshold is chosen a lower HR is achieved, but the percentage of patients included in class 1 is also far lower (FIG. 3c). This shows it is essential to have a lower limit for the size of class 1 and not solely consider HR. In the training set 28.4% of patients are classified a class 1, resulting in an HR of 0.13

(p=7.1*10-11) between the two treatment arms (FIG. 3c). More importantly, in the test set (fold D1) an HR of 0.47 (p=0.03) is achieved with 27.0% of patients classified as class 1 (FIG. 3d). This demonstrates that the classifier successfully validates on new and unseen data, a feat not achieved with any currently existing marker.

TOPSPIN is Successfully Validated on Fold D2 and D3

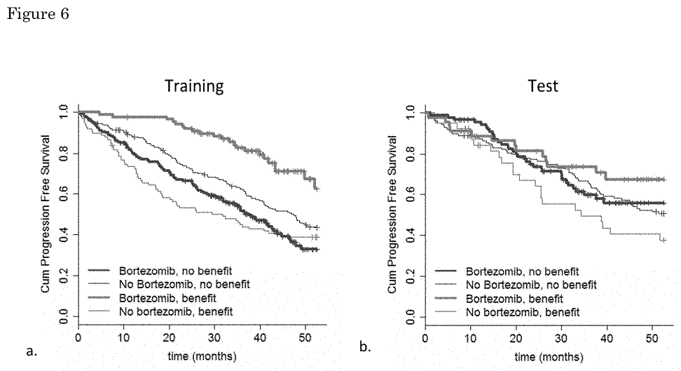

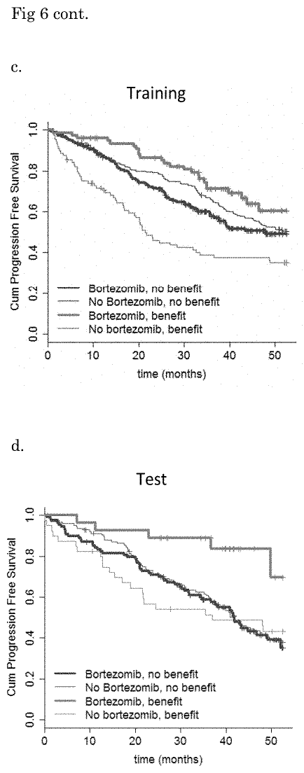

Fold D1 was kept entirely separate during the development of the algorithm. After validation of the final classifier on fold D1, further validation of the robustness of the approach was sought by rotating the validation folds D2 and D3. In these cases, fold D1+D3 and fold D1+D2 were used as training data, respectively. These classifiers also validated, with even lower p-values, and resulted in an HR of 0.42 (p=0.02) on fold D2 and an HR of 0.28 (p=0.01) on fold D3 (Table 2). Kaplan Meier curves of training and test classifications are shown in FIG. 6.

Gene Ontology Sets are Important for Performance

To assess whether the performance of the final classifier is due to the biology captured by the Gene Ontology, the experiment was repeated using random gene sets. That is, the number of genes in each set were kept the same, but the identity of the genes was random. Again the top 8 performing gene sets are used in the final classifier. The optimal threshold found is t=4. In the training set, 20.0% of patients are classified as class 1, with an HR of 0.10 (p=7.4*10-10). However, in the test set this HR did not remain significant (22.0% of patients are classified as class 1, with an HR of 0.73 (p=0.38)) (Table 2). Thus, even though the training performance is comparable to that of the classifier using GO sets, the classifier built from the random gene sets does not validate, indicating that information on functional relation between genes is important for classifier construction.

Prediction of TOPSPIN-Derived Labels Directly

We also asked whether the labeling found with TOPSPIN is sufficient to obtain treatment-specific survival prediction when combined with a standard state-of-the-art classifiers. To this end, the nearest mean was trained, random forest and support vector machine classifiers directly on the gene expression data using the labels found by TOPSPIN. The results are summarized in Table 3 and demonstrate that none of the classifiers achieve a significant HR in the test set. Interestingly, the nearest mean classifier and the support vector machine do achieve a lower HR than the baseline HR. The class 1 identified by the support vector machine classifier is relatively small and thus may lack power to achieve statistical significance. However, the HR of 0.33 is promising and is substantially lower than the HR achieved by the support vector machine when trained on labels straightforwardly derived from the survival data. This may indicate that the labels found by TOPSPIN identify a more clinically relevant subset of patients. That said, the built-in classifier of TOPSPIN is still required to obtain a sufficiently large class 1 and statistically significant HR.

TOPSPIN Outperforms Known Markers

We finally compared the predictive performance of TOPSPIN to the known prognostic markers del17p and RPL5 that were found to also have predictive value (Neben et al., 2012; Hofman et al., 2015). For the chromosomal aberration del17p, data was only available for the HOVON65/GMMG-HD4 dataset. Patients for which the aberration is present were classified as class 1 and all others were classified as class 0. This resulted in a substantially poorer HR of 0.52 (p=0.18). Moreover, with only 13.6% of patients classified as class 1, the del17p marker provides a treatment decision for a smaller proportion of patients than the TOPSPIN classifier. For the RPL5 marker, the 22.5% of patients with lowest expression for this gene are defined as class 1, i.e., benefiting from bortezomib. This threshold was defined on the HOVON65/GMMG-HD4 dataset in the original paper, so these patients were excluded when this marker was tested on the dataset. This resulted in an HR of 0.70 (p=0.22) in class 1 (22.5%). Taken together, it is clear that TOPSPIN identifies a more relevant way of identifying a subset of patients with a substantially larger benefit from bortezomib than any of the known markers.

Overlap Between the Genes Used in the Different Classifiers

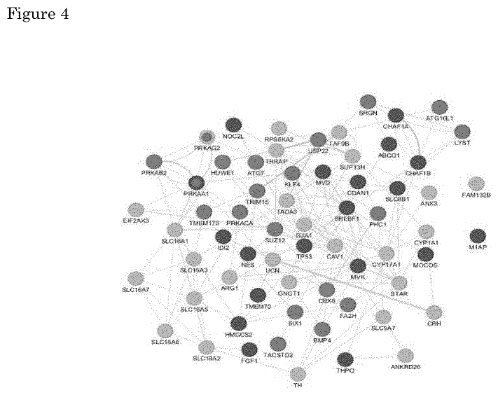

All genes used in the classifier to predict folds D1 (red nodes), D2 (purple nodes) and D3 (blue nodes) are shown in FIG. 4. The edges between the nodes indicate a functional relationship or physical interaction provided by the GeneMANIA algorithm (Warde-Farley et al, 2010). GeneMANIA mines a large set of functional association data to provide connections between genes, as indicated by the edges in FIG. 4. This provides an overview of how the genes selected in the classifier relate to each other.

We observe two genes that are shared among at least two independent classifiers. This relatively small number is unsurprising, since it has been shown before that many different sets of genes can have a comparable performance in a classification task (Ein-Dor et al., 2005). PRKAA1 is a subunit of the AMPK complex and has previously been reported to be associated with prognosis in colorectal cancer (Lee et al., 2014). PRKAG1 is the γ subunit of AMPK and has also been linked colon cancer cell survival (Fisher et al., 2015). Interestingly, through these genes, all three classifiers use genes associated with the AMPK complex. AMPK is a metabolic stress sensor and plays an important role in controlling cellular growth. It has also been suggested to play a role in recognizing genomic stress and DNA damage response (Sanli et al., 2014), suggestive of a role in determining treatment response. The network also includes a number of other genes that are well known to regulate the cell cycle and control proliferation, like TP53 (Lane, 1992). The deubiquitination enzyme USP22 has previously been reported to be associated with prognosis and tumor progression in hepatocellular carcinoma (Tang et al, 2015), non-small cell lung cancer (Hu et al., 2015), and colorectal cancer and it has been suggested USP22 plays a central role in cell cycle progression (Liu et al., 2012). Because bortezomib is a proteasome inhibitor, which triggers apoptosis through the unfolded protein response when ubiquitinated proteins accumulate in the cell (Obeng et al, 2006), it is plausible that a deubiquitination enzyme plays a role in bortezomib response.

Finally, the network representation clearly demonstrates that the genes used in each classifier are not only connected with each other, but also between classifiers. So, while TOPSPIN does not consistently use the same genes to predict bortezomib benefit, it does use genes with similar functions that are known to be involved in cell cycle regulation and cancer development. This finding is in line with the observation that a superior performance is obtained when GO gene sets are used compared to random gene sets.

### Discussion

Here, TOPSPIN is proposed, a novel classifier for treatment-specific outcome prediction. The utility of TOPSPIN on a combination of phase III multiple myeloma clinical trial datasets was demonstrated. It has been found that TOPSPIN can successfully identify and predict the subset of patients that will benefit from bortezomib. The final classifier is validated on independent data that was left out of the development phase completely to prevent experimenter bias. This validation resulted in a significant HR (p=0.03), which is much more significant when compared to existing markers. Moreover, only good performance was observed while incorporating functional information between genes, encoded in the GO.

We compared TOPSPIN to standard linear and non-linear binary classifiers trained on labels straightforwardly derived from the survival data. Without exception, poor validation performance was observed. In contrast, when TOPSPIN labels were trained on directly, some performance on the validation fold was not seen. This demonstrates that the way in which TOPSPIN determines target labels, by using genetic similarity between patients that receive opposite treatment, is an important ingredient for successful treatment-specific prediction. Nevertheless, the best performance was observed when also including the classification employed in TOPSPIN. Taken together, it is concluded that the way in which TOPSPIN defines labels, in combination with the prototype-based classification, is important for finding the most clinically relevant subset of patients.

We note that TOPSPIN is a generic method that can be used on any dataset with two randomized treatment arms and a continuous outcome measure. Therefore, TOPSPIN will be an important post-hoc analysis for phase III clinical trials of novel treatments that have missed their endpoint, such as, for instance, nivolumab in the CheckMate-026 trial (Socinki et al., 2016). Considering the often low response rates combined with the serious side effects of current cancer therapies, TOPSPIN therefore offers an important step toward realistic personalization of cancer medicine.

### REFERENCES

Aressy, B. et al. (2010) A screen for deubiquitinating enzymes involved in the G2/M checkpoint identifies USP50 as a regulator of HSP90-dependent Weel stability. Cell Cycle, 9, 3839-3846, doi: 10.4161/cc.9.18.13133
Barlogie, B., et al. (2006). Total therapy 2 without thalidomide in comparison with total therapy 1: Role of intensified induction and posttransplantation consolidation therapies. Blood, 107, 2633-2638. doi:10.1182/blood-2005-10-4084
Barlogie, B., et al. (2007). Incorporating bortezomib into upfront treatment for multiple myeloma: Early results of total therapy 3. British Journal of Haematology, 138, 176-185. doi:10.1111/j.1365-2141.2007.06639.x
Block, K. I., et al. (2015). Designing a broad-spectrum integrative approach for cancer prevention and treatment. Seminars in Cancer Biology, 35, S276-S304. doi:10.1016/j.semcancer.2015.09.007
Burrell, R. A., et al. (2013). The causes and consequences of genetic. Nature, 501, 338-345. doi:10.1038/nature12625
Carlson M (2016). hgu133plus2.db: Affymetrix Human Genome U133 Plus 2.0 Array annotation data (chip hgu133plus2). R package version 3.0.0.
Davis, R. E., & Staudt, L. M. (2002). Molecular diagnosis of lymphoid malignan-cies by gene expression profiling. Current Opinion in Hematology, 9, 333-8. doi:10.1097/01.MOH.0000015263.68414.07
Ein-Dor, L., et al. (2005). Outcome signature genes in breast cancer: Is there a unique set? Bioinformatics, 21, 171-178. doi:10.1093/bioinformatics/bth469
Fisher, K. W. et al. (2015). AMPK Promotes Aberrant PGC1β Expression To Support Human Colon Tumor Cell Survival. Molecular and Cellular Biology, 35, 3866-3879. doi:10.1128/MCB.00528-15
Hofman, I. J. F., et al. (2015). RPL5 Is a Candidate Tumor Suppressor on 1p22.1 in Multiple Myeloma of Which the Expression Is Linked to Bortezomib Re-sponse. Blood, 126, 2969 LP-2969.
Howlader N, et al. (2016). SEER Cancer Statistics Review, 1975-2013. In National Cancer Institute. Bethesda, Md. Retrieved from http://seer.cancer.gov/csr/1975 2013/
Hu, J., et al. (2015). USP22 promotes tumor progression and induces epithelial-mesenchymal transition in lung adenocarcinoma. Lung Cancer, 88, 239-245. doi:10.1016/j.lungcan.2015.02.019
Johnson, W. E., Li, C., & Rabinovic, A. (2007). Adjusting batch effects in microarray expression data using empirical Bayes methods. Biostatistics (Oxford, England), 8, 118-27. doi:10.1093/biostatistics/kxj037
Keats, J. J., et al. (2012). Clonal competition with alternating dominance in multiple myeloma. Blood, 120, 1067-1076. doi:10.1182/blood-2012-01-405985
Kumar, S. K., et al. (2008). Improved survival in multiple myeloma and the impact of novel therapies. Blood, 111, 2516-2520. doi:10.1182/blood-2007-10-116129
Lane, D P. (1992) P53, the guardian of the genome. Nature. 358: 15-16.
Lee, S. J., et al. (2014). Genetic variations in STK11, PRKAA1, and TSC1 associ-ated with prognosis for patients with colorectal cancer. Ann. Surg. Oncol., 21, S634-9. doi:10.1245/s10434-014-3729-z
Liaw, A. and Wiener, M. (2002). Classification and Regression by randomForest. R News 2(3), 18-22.
Lièvre, A., et al. (2006). KRAS mutation status is predictive of response to cetuximab therapy in colorectal cancer. Cancer Research, 66, 3992-3995. doi:10.1158/0008-5472.CAN-06-0191
Liu, Y. L. et al. (2012). USP22 Acts as an Oncogene by the Activation of BMI-1-Mediated INK4a/ARF Pathway and Akt Pathway. Cell Biochemistry and Biophysics, 62, 229-235. doi:10.1007/s12013-011-9287-0
Lohr, J. G., et al. (2014). Widespread genetic heterogeneity in multiple myeloma: Implications for targeted therapy. Cancer Cell, 25, 91-101. doi:10.1016/j.ccr.2013.12.015
Meyer, D. et al., (2015). e1071: Misc Functions of the Department of Statistics, Probability Theory Group (Formerly: E1071), TU Wien. R package versi-on 1.6-7. http://CRAN.R-project.org/package=e1071
Molina, M. A., et al. (2001). Trastuzumab (Herceptin), a humanized anti-HER2 receptor monoclonal antibody, inhibits basal and activated HER2 ectodomain cleavage in breast cancer cells. Cancer Research, 61, 4744-4749. doi:11406546
Munshi, N. C., & Anderson, K. C. (2013). New strategies in the treatment of multiple myeloma. Clinical Cancer Research: An Official Journal of the American Association for Cancer Research, 19, 3337-44. doi:10.1158/1078-0432.CCR-12-1881
Neben, K., et al. (2012). Administration of bortezomib before and after autologous stem cell transplantation improves outcome in multiple myeloma patients with deletion 17p. Blood, 119, 940-948. doi:10.1182/blood-2011-09-379164
Obeng, E. et al. (2006). Proteasome inhibitors induce a terminal unfolded protein response in multiple myeloma cells. Blood, 107, 4907-4916. doi:10.1182/blood-2005-08-3531
Palomero, T., Lim, W. K., Odom, D. T., Sulis, M. L., Real, P. J., Margolin, A., . . . Ferrando, A. a. (2006). NOTCH1 directly regulates c-MYC and activates a feed-forward-loop transcriptional network promoting leukemic cell growth. Proceedings of the National Academy of Sciences of the United States of America, 103, 18261-18266. doi:10.1073/pnas.0606108103
Prasad, V. (2016). Perspective: The precision-oncology illusion. Nature, 537, S63-S63. doi:10.1038/537S63a
Sanli, T., Steinberg, G. R., Singh, G., & Tsakiridis, T. (2014). AMP-activated protein kinase (AMPK) beyond metabolism: a novel genomic stress sensor participating in the DNA damage response pathway. Cancer Biology & Therapy. doi:10.4161/cbt.26726
Santos, C., et al. (2015). Intrinsic cancer subtypes-next steps into personalized medicine. Cellular Oncology, 38, 3-16. doi:10.1007/s13402-014-0203-7
Socinki et al. (2016). CheckMate 026: A phase 3 trial of nivolumab vs investiga-tor's choice (IC) of platinum-based doublet chemotherapy (PT-DC) as first-line therapy for stage iv/recurrent programmed death ligand 1 (PD-L1)-positive NSCLC. Ann Oncology, 27, Suppl_6:LBA7_PR
Sonneveld, P. et al. (2013) Bortezomib-based versus nonbortezomib-based induction treatment before autologous stem-cell transplantaion in patients with previously untreated Multiple Myeloma: A meta-analysis of phase III randomized, controlled trials. Journal of Clinical Oncology. 31, 3279-3287.
Tang, B. et al. (2015). High USP22 expression indicates poor prognosis in hepato-cellular carcinoma. Oncotarget, 6, 12654-12667. doi:10.18632/oncotarget.3705
Therneau T (2015). A Package for Survival Analysis in S. version 2.38, https://CRAN.R-project.org/package=survival.
Tsao, M.-S., et al. (2005). Erlotinib in lung cancer—molecular and clinical predic-tors of outcome. The New England Journal of Medicine, 353, 133-144. doi:10.1056/NEJMoa050736
Van 't Veer, L. J., et al. (2002). Gene expression profiling predicts clinical outcome of breast cancer. Nature, 415(6871), 530-536. Retrieved from http://dx.doi.org/10.1038/415530a
Warde-Farley, et al. (2010). The GeneMANIA prediction server: Biological network integration for gene prioritization and predicting gene function. Nucleic Acids Research, 38. doi:10.1093/nar/gkq537

### Example 2

Methods

Data and processing; endpoint and survival analysis; and gene sets was carried out as described in Example 1.

Algorithm

The algorithm was similar as, for example, 1 except for minor changes discussed below.

STL classifier/TOPSPIN aims to predict if a patient does or does not benefit from a certain treatment of interest based on the gene expression profile of the patient. In order to train this classifier, a gene expression dataset is required that consists of two treatment arms and a continuous outcome measure. These data are first split into training and validation folds. The training data comprises two thirds of the data, while one third (fold D) is kept apart to function as validation data. Three separate folds are defined D (D1, D2 and D3), such that each patient is included in the validation set once. The training data is subsequently split further into folds A, B and C for training.

We first define a ranked list of prototype patients on fold A (Step 1) that exhibit a better than expected prognosis on the treatment of interest compared to a set of genetically similar patients that received an alternative treatment. In Step 2, a decision boundary around a selection of prototype patients is determined on fold B. Patients that lie within this decision boundary are expected to show a favorable outcome when receiving the treatment of interest and are classified as benefitting (class ‘benefit’). All other patients are considered class ‘no benefit’ and are not expected to benefit from receiving the treatment of interest. Because it is a priori unknown based on which genes patient similarity should be defined, step 1 and 2 are performed for a large number of functionally coherent gene sets obtained from the Gene Ontology annotation, yielding one classifier per gene set. Step 1 and 2 are repeated 12 times to obtain a robust estimate of the performance per gene set. In each repeat, the training data is split into a different fold A, B and C. The performance is defined as the Hazard Ratio (HR) between treatments in class ‘benefit’, found in a fold C, which contains samples that were not used in step 1 and 2. All gene sets are ranked by their mean performance in fold C across repeats. In Step 3 the optimal number of gene sets to combine into a final classifier is determined. It is found that defining performance and selecting the optimal number of gene sets on the same folds C leads to overtraining. Therefore, the entire algorithm is run a second time (Run 2), using 12 new repeats with different splits into fold A, B and C. The first run of 12 repeats is used to rank the gene sets. The combined performance of these ranked gene sets on the folds C from Run 2 is used to determine the optimal number s of gene sets. Similar to the boosting principle³⁵, the individual classifiers are combined into an ensemble to construct a more robust final classifier. The performance of this combined classifier is measured on fold C of Run 2. The gene sets are added to the classifier in order of their ranking, until an optimal performance is reached across all the repeats from Run 2. Since there are 12 repeats, each combination results in 12 HRs as measured on the folds C from run 12. To determine the optimal number of gene sets, a local polynomial regression line is fit on the median HRs for each combination of gene sets. The optimal number of gene sets k is reached when adding a gene set does not result in a lower HR. The gene sets are then ranked based on their individual performance across the folds C of Run 2 and select the top k for inclusion in the final ensemble classifier. Finally, in Step 4, one final classifier is trained using the entire training dataset for these selected gene sets.

These steps are visualized in FIG. 7d and are described in more detail below.

Step 1—Prototype Ranking on Fold A

For each patient receiving the treatment of interest, the treatment benefit is defined as

$${PFS}_{i} = \frac{1}{n} {\sum }_{j ∈ O} ( {PFS}_{i} - {PFS}_{j} ) ,$$

where O is the set of the n most similar patients (based on Euclidean distance) that did not receive the treatment of interest. n=10 is used. ΔPFS is only calculated for neighbor pairs where it is clear which patient experienced an event first; if both are censored or one patient is censored before the neighbor experienced an event, ΔPFS is not computed. To correct for the fact that a patient with a long survival time will, on average, have a large ΔPFS irrespective of its relative treatment benefit compared to genetically similar patients, the z-normalized zPFS score is defined as:

$${zPFS}_{i} = \frac{Δ {PFS}_{i} - μ ( {RPFS}_{i} )}{σ ( {RPFS}_{i} )} ,$$

where RPFS is a distribution of 1000 random ΔPFS scores, obtained by calculating ΔPFS for randomly chosen sets O, i.e., determining treatment benefit with respect to random patients instead of genetically similar patients. Based on the zPFS score all patients in fold A that were given the treatment of interest can be ranked.

Step 2—Classifier Definition on Fold B

The classifier is defined by a subset of z top-ranked prototypes along with a decision boundary defined in terms of the Euclidean distance γ around a prototype. A patient is classified as class ‘benefit’ when it lies within γ of any of the top z prototypes. The optimal values for z and γ are those resulting in the lowest Hazard Ratio (HR) in class ‘benefit’ (the patient group in which the treatment of interest should have a better survival). An operating point is set that additionally constrains k and γ, such that class ‘benefit’ comprises at least a certain percentage of the dataset. This ensures sufficient statistical power to compute the significance of the HR in the ‘benefit’ class. The number of prototypes was restricted to 10 to prevent defining an extremely complicated classifier. The search grid for parameter γ was made dependent on the local density of the neighbors, and consisted of the sorted list of Euclidean distances between the prototype and its neighbors. The optimal z and γ combination is chosen so that the HR in class ‘benefit’ is minimal, while still associated with a p-value below 0.05. If no combination results in a p-value below 0.05, the minimal non-significant HR is chosen.

Step 3—Rank and Select Gene Sets

The gene sets are ranked by their mean performance in fold C over all repeats from Run 1. After ranking, the algorithm is run a second time, with different divisions into fold A, B and C. Gene sets are added to an ensemble classifier one by one based on this ranking. The performance of the combined gene sets is measured on each fold C of this second run. It is found that defining the ranking on different folds than is used to measure combined performance prevents overtraining, although some bias is still expected to occur. Since the found HR can fluctuate between folds and gene set numbers, a regression line is fit through the median HRs found on folds C in the second run and the optimal number of gene sets is determined: the first combination of gene sets for which adding another gene set does not lead to an improvement of the HR larger than 1*10⁻⁴.

Step 4—Build Final Classifier

After the optimal number of gene sets is determined in Step 3, the gene sets are ranked based on their mean performance in fold C in the second run. The top scoring gene sets are selected and for these gene sets a final classifier is trained. To this end, the complete training dataset is split into only two folds, since the third fold is no longer required. The classifiers defined by different gene sets are combined into an ensemble classifier by an equally weighted voting procedure, which means each classifier has an equal influence on the final classification. For an ensemble classifier containing k gene sets, this defines a classification score between 0 and k per patient. This score is thresholded by threshold T, which determines whether a patient is to benefit from the treatment of interest, where a patient with a score below the threshold is classified as not benefitting from treatment (no ‘benefit’ class). The optimal threshold T is the one for which the HR between treatments is minimal in class ‘benefit’. This combination of classifiers and threshold can be used to classify new and unseen patients and is validated on fold D.

Calculating Overrepresentation of Genes Used in the Classifier

The same gene can be used multiple times in a single classifier and/or multiple times across the classifiers obtained for fold D1, D2 and D3. Both cases provide evidence of the importance of the gene for the treatment benefit prediction. To assess whether genes are selected more frequently than expected by chance across all three classifiers, it is determined the degree of overrepresentation by dividing the observed count by the expected count. The expected count is calculated by p*W where p is the fraction of the gene sets containing the gene and W the total number of gene sets selected across all three classifiers. A p-value is determined using the binomial test.

Training Regular Classifiers

We defined the labels that were used to train the regular classifiers in two ways. First, labels were defined by assigning the 25% longest surviving bortezomib patients and the 25% shortest surviving non-bortezomib patients to the ‘benefit’ class and all others to the ‘no benefit’ class. A classifier was trained using folds A-C to predict these labels, using the HR in validation fold D1 as performance measure of the predictive power. For the nearest mean classifier, a double-loop cross-validation was used to optimize the number of genes (ranked based on t-score), using balanced accuracy as the performance measure.

A random forest classifier (R package randomForest, version 4.6.12)³⁶ and a support vector machine (R package e1071, version 1.6.7)³′ were also trained. For both these classifiers, the number of genes was optimized in cross validation. For the random forest classifier 2000 trees were trained per classifier and the bootstrap sample was sampled equally from both classes, to prevent the classifier being affected by the class imbalance. For the support vector machine, C-values from 1 to 100 were tested, in steps of 1. The gamma used is 1/P, where P is the number of input variables, i.e., the number of genes.

For all classifiers, the accuracy reported is the mean accuracy in cross validation for the optimal number of input genes.

Comparison with Known Prognostic Markers

To the best of our knowledge, RPL5 is the only published gene expression based marker that predicts bortezomib benefit by comparing to another treatment group¹⁸. RPL5 is tested on the data from the Total Therapy studies, since it was trained on the HOVON-65 data. Since some predictive markers are discovered by testing markers previously known to be prognostic, prognostic markers are also compared. FISH markers were called on the gene expression data, using previously developed classifiers³⁸, since FISH data was not available for all patients. Unfortunately, there is no reliable gene expression classifier for del17p. Any predictive information that was available in previously defined molecular subtypes in MA³⁹ and in the prognostic gene signature EMC-92⁴⁰ was also tested.

Code Availability

All code needed to train and validate the classifier is available at github.com/jubels/GESTURE.

### Results

We combined data from three randomized phase III clinical trials comprising of 910 patients with MM (see methods), who either received the proteasome inhibitor bortezomib (n=407) or not (n=503). For each patient gene expression profiles were generated from purified myeloma plasma cells at diagnosis. An overall HR of 0.74 (p=0.0029) is observed between the two treatment arms, in favor of the bortezomib arm. While this HR indicates significant treatment benefit for bortezomib, it was asked whether this was driven by a small benefit for all patients, or if a subgroup of patients can be identified showing a large benefit from treatment with bortezomib, while the remainder of patients show a smaller or no benefit from bortezomib. This research aims to identify a subset of patients, the ‘benefit’ class, who benefit from the treatment of interest (bortezomib) relative to a comparator treatment arm that does not contain bortezomib. The patients not included in the ‘benefit’ class belong to the class ‘no benefit’ and would not benefit from receiving bortezomib. The classifier identifying this ‘benefit’ class could serve as a valuable diagnostic to determine which newly diagnosed patients would benefit from bortezomib (based) treatment.

The key idea of STL is that a patient's treatment benefit can be estimated by comparing its survival to a set of genetically similar patients that received the comparator treatment (FIG. 7d, step 1). Patients with a large survival difference compared to genetically similar patients can then act as prototype patients; new patients with a similar gene expression profile are expected to also benefit from receiving the treatment of interest. Since similarity in gene expression profile is greatly influenced by the choice of input genes, This similarity is defined according to a large number of gene sets. Training the prototype-based classifier requires optimizing two parameters per gene set: the number of prototypes to use and the decision boundary, defined in terms of the Euclidean distance to the prototype (FIG. 7d, step 2). The STL classifier also needs to select the optimal gene sets to ultimately classify a patient. Importantly, the labels are now defined using the prototypes identified for the various gene sets, which means that in the STL approach there is no need to define labels before training the classifier. To train the classifier and select the best performing gene sets, the training data are split in three folds (A, B and C). Fold A is used to identify prototypes, fold B to optimize the decision boundary and fold C to estimate classifier performance.

To obtain unbiased estimates of the overall prediction performance, the entire dataset is divided in three equal folds, D1, D2 and D3, ensuring a similar HR between the treatment arms in all three folds. Training is performed on two folds, while the remaining fold is kept separate to serve as an independent validation set. This is rotated to obtain an unbiased prediction for each fold. The division of the data in D1, D2 and D3, and subsequently in folds A, B and C is shown in FIG. 7c.

It is a priori unknown which genes will be relevant to defining patient similarity and predicting treatment response. 10,581 functionally coherent gene sets were used based on Gene Ontology annotation. Each gene set is used to train a separate classifier. The top-performing classifiers are subsequently combined into an ensemble classifier to determine the optimal number of gene sets to be used in the final classifier (FIG. 7d, step 3, for details see Methods). For the gene sets included in this optimal number a single classifier is trained using all the training data. These classifiers are combined into the final ensemble classifier that is used to classify the patients in the validation set (FIG. 7d, step 4).

Simulated Treatment Learning Identifies a Predictive Classifier for Bortezomib Benefit

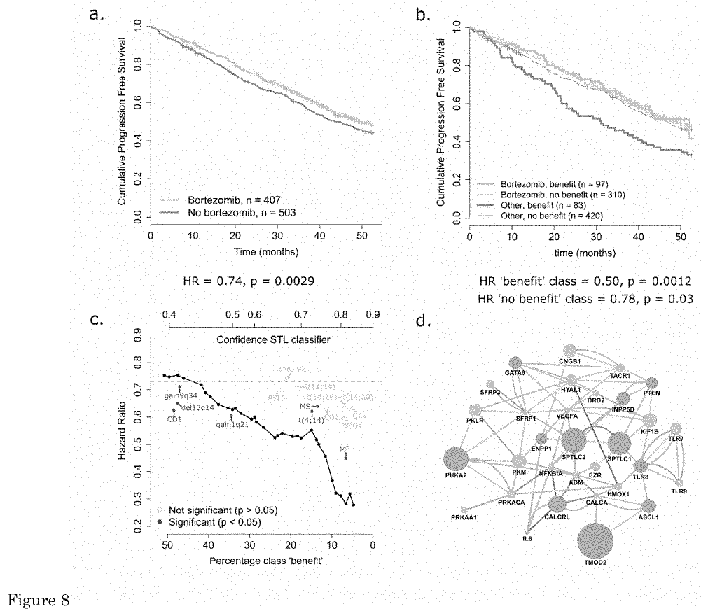

FIG. 8a shows the cumulative progression free survival curves for two treatment arms, with an HR of 0.74 (p=0.0029) between the treatment arms. FIG. 8 shows the treatment arms and classes as identified by the STL classifier. The HRs for the ‘benefit’ and ‘no benefit’ class are 0.50 (p=0.0012) and 0.78 (p=0.03), respectively. These results show that a subgroup, comprising 19.8% of the population (n=180), benefits substantially more from bortezomib treatment than the population as a whole. More importantly, the STL approach is able to discover and predict this subgroup using the gene expression data at diagnosis.

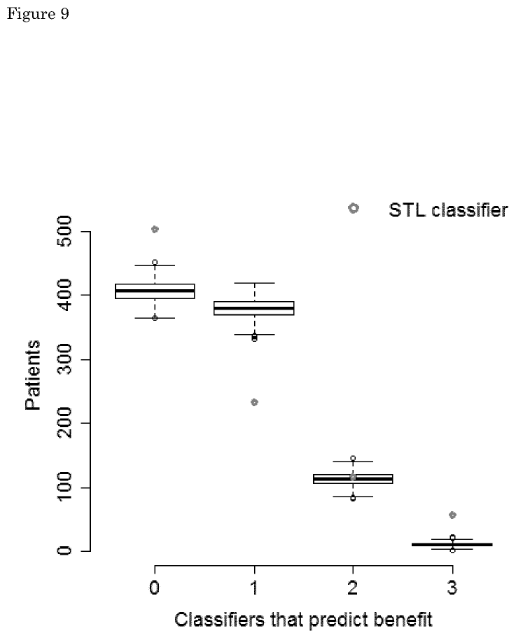

The HR of 0.50 results from combining the classifications in individual folds. The results obtained within each of the individual validation folds are similar to this HR, reaching an HR of 0.51 (p=0.03), 0.39 (p=0.07) and 0.46 (p=0.06) in folds D1, D2 and D3 respectively. The effect was comparable in all folds, demonstrating a stable performance, although not statistically significant for fold D2 and D3 at p<0.05 due to the fact that in D2 9.9% of patients and in D3 20.1% are included in the ‘benefit’ class and versus 29.4% in D1. Although there are no ground truth labels available, the class labels obtained are compared with the three separate classifiers when applied to all 910 patients. It is found that these three class assignments agree between the classifiers significantly more than expected by chance (FIG. 9) A similar conclusion is reached by comparing the classification scores directly, which significantly correlate (maximum p<1*10⁻⁴). When considering the cases for which the three classifiers agree, it is found that 503 patients are consistently classified as ‘no benefit’ and 57 patients as ‘benefit’. Together, this demonstrates that, even though the classifiers do not agree on the class assignment for all patients, they capture the same gene expression patterns.

The operating point of the classier is determined by the number of individual classifiers in the ensemble that agree on the class label, and is thus directly related to the confidence of the ensemble classifier about the label ‘benefit’. To ensure sufficient power and provide a treatment decision for a substantial group of patients, the operating point of the classifier was set to 20% in training (see methods). At this operating point, 19.8% of patients in the validation folds were actually assigned to the ‘benefit’ class. FIG. 8c depicts the HR as a function of the confidence level of the classifier. It is observed that, for higher confidence levels (yielding smaller sizes of the ‘benefit’ class) more extreme validation HRs are observed, demonstrating that there is a direct relation between classifier score and treatment benefit. This is consistent with the fact that the highest HR and largest class ‘benefit’ are found in fold D1 in validation, while the lowest HR and the smallest class ‘benefit’ are found in D2.

STL Classifier Outperforms Known Markers

We compared the HRs found using the STL classifier with several known prognostic markers in MM, some of which also show predictive value (FIG. 8c). The STL classifier has a superior performance for operating points that result in assignment of up to 30% of the patients to the class ‘benefit’. The markers that slightly outperform the STL classifier do so only for operating points that results in much larger sizes of the class ‘benefit’ and lead to smaller effect sizes. The grey line indicates the baseline HR found in the entire dataset. A clinically actionable classifier should find a substantially larger benefit than this baseline, which is only attained by the STL classifier for operating points <30%.

Biological Information is Important for Performance

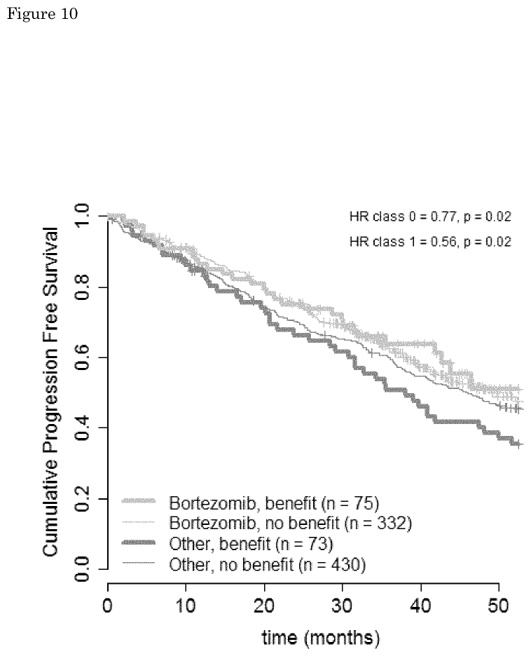

To investigate if the biological knowledge contained in the Gene Ontology, used to define gene sets, truly aids classification performance, random gene sets were also tested with the same set size distribution. Using the random gene sets, final classification results in a significant HR of 0.56 (p=0.02) when all three validation folds are combined (FIG. 10). This is not unexpected as combining random feature sets in an ensemble classifier is known to achieve good classification performance¹⁹. Moreover, it has been shown previously that random gene signatures can perform on par in a prognostic setting²⁰. Nonetheless, the STL classifier trained using the GO gene sets outperforms the random gene set approach in both HR and p-value. Moreover, in contrast to the relatively stable performance across validation folds when using the GO gene sets, the performance of the random set approach varies greatly between the folds, ranging from an HR of 0.76 (p=0.55) in D1 to an HR of 0.44 (p=0.03) in D3. Together, this demonstrates that the biological information contained in the Gene Ontology gene sets is important to the performance of the STL classifier.

Genes Used to Predict Treatment Benefit Bortezomib

The classifiers built for D1, D2 and D3 use respectively 113, 218 and 111 GO gene sets to predict bortezomib benefit, encompassing a total of 1913 unique genes. There are 31 genes used in all three classifiers (FIG. 8d). There are GO categories that include a large subset of these 31 genes, including “positive regulation of transcription from RNA polymerase II promoter”, “cellular response to hypoxia” and “negative regulation of the apoptotic process”. All these GO categories are associated with the pathogenesis of cancer. Both increased proliferation and the ability to evade apoptosis are hallmarks of cancer²¹. It has also been established that cancer cells can adapt their metabolism to thrive in hypoxic conditions²². For the 31 genes, it is calculated that they are selected more than expected by chance. The expected count for a gene is based on the number of GO categories that include that gene, e.g., PTEN is included in 123 of the 10,581 gene sets, so in the 442 gene sets used across D1, D2 and D3 it is expected to have observed PTEN approximately 5 times if it would occur at the same frequency as within the selected gene sets. Most genes in common between the three classifiers are observed more often than expected (degree of overrepresentation indicated by node size in FIG. 8d), with 11 of 31 significantly overrepresented (p<0.05). The most overrepresented genes are TMOD2, PHKA2, SPTCL1 and SPTCL2. None of these genes are known to be associated with MM or response to bortezomib. However, investigation of the proteome of a cell line carrying a SPTCL1 mutation showed an increased presence of Ig kappa chain C²³. Immunoglobulin light chain presence is used as a biomarker for MM and has been identified as a risk factor for progression²⁴. PTEN is also found to be significantly overrepresented. PTEN is a known tumor suppressor and was found to be mutated in a various cancers²⁵. In MM, PTEN mutations are relatively uncommon and associated with advanced disease²⁶.

### REFERENCES

1. Burrell, R. A., McGranahan N., Bartek, J. and Swanton, C. The causes and consequences of genetic heterogeneity in cancer evolution. Nature, 501, 338-345. (2013).
2. Block, K. I., et al. Designing a broad-spectrum integrative approach for cancer prevention and treatment. Seminars in Cancer Biology, 35, S276-S304. doi:10.1016/j.semcancer.2015.09.007 (2015)
3. Santos, C., et al. Intrinsic cancer subtypes-next steps into personalized medicine. Cellular Oncology, 38, 3-16. (2015).
4. Lièvre, A., et al. KRAS mutation status is predictive of response to cetuximab therapy in colorectal cancer. Cancer Research, 66, 3992-3995. (2006).
5. Bernard, P. S., et al. Supervised risk predictor of breast cancer based on intrinsic subtypes. Journal of Clinical Oncology, 27, 1160-1167. (2009).
6. Walther, A. Genetic prognostic and predictive markers in colorectal cancer. Nature Reviews Cancer, 9, 489-499. doi:10.1038/nrc2645 (2009).
7. Van 't Veer, L. J., et al. Gene expression profiling predicts clinical outcome of breast cancer. Nature, 415(6871), 530-536. 12002).
8. Cardoso, F. et al. 70-Gene Signature as an Aid to Treatment Decisions in Early-Stage Breast Cancer. New England Journal of Medicine, 375, 717-729. doi:10.1056/NEJMoa1602253 (2016).
9. Bhutani, M., et al. Investigation of a gene signature to predict response to immunomodulatory derivatives for patients with multiple myeloma: an exploratory, retrospective study using microarray datasets from prospective clinical trials. The Lancet Haematology, 4, e443-e451. doi:10.1016/52352-3026(17)30143-6 (2017).
10. Vangsted et al., Drug response prediction in high-risk multiple myeloma. Gene (2017).
11. Ting, K. R., et al. Novel panel of protein biomarkers to predict response to bortezomib-containing induction regimens in multiple myeloma patients. BBA Clinical, 8, 28-34. http://doi.org/10.1016/j.bbacli.2017.05.003 (2017).
12. Howlader N, et al. SEER Cancer Statistics Review, 1975-2013. In National Cancer Institute. Bethesda, Md. Retrieved from http://seer.cancer.gov/csr/1975_2013/(2016).
13. Kumar, S. K., et al. Improved survival in multiple myeloma and the impact of novel therapies. Blood, 111, 2516-2520. doi:10.1182/blood-2007-10-116129 (2008)
14. Munshi, N. C., & Anderson, K. C. New strategies in the treatment of multiple myeloma. Clinical Cancer Research, 19, 3337-44. doi:10.1158/1078-0432.CCR-12-1881 (2013).
15. Lohr, J. G., et al. Widespread genetic heterogeneity in multiple myeloma: Implications for targeted therapy. Cancer Cell, 25, 91-101. doi:10.1016/j.ccr.2013.12.015 (2014).
16. Keats, J. J., et al. Clonal competition with alternating dominance in multiple myeloma. Blood, 120, 1067-1076. doi:10.1182/blood-2012-01-405985 (2012).
17. Neben, K., et al. Administration of bortezomib before and after autologous stem cell transplantation improves outcome in multiple myeloma patients with deletion 17p. Blood, 119, 940-948. doi:10.1182/blood-2011-09-379164 (2012).
18. Hofman, I. J. F., et al. RPL5 on 1p22.1 is recurrently deleted in multiple myeloma and its expression is linked to bortezomib response. Leukemia, 31(8), 1706-1714. http://doi.org/10.1038/leu.2016.370 (2017).
19. Breiman, L. Random Forests. Machine Learning, 45(1), 5-32. http://doi.org/10.1023/A:1010933404324 (2001).
20. Venet, D., Dumont, J. E., and Detours, V. Most random gene expression signatures are significantly associated with breast cancer outcome. PLoS Computational Biology, 7(10). http://doi.org/10.1371/journal.pcbi.1002240 (2011).
21. Hanahan, D., & Weinberg, R. A. Review Hallmarks of Cancer: The Next Generation. Cell, 144(5), 646-674. http://doi.org/10.1016/j.cell.2011.02.013 (2011).
22. Eales, K. L., Hollinshead, K. E. R., and Tennant, D. A. Hypoxia and metabolic adaptation of cancer cells. Oncogenesis, 5, e190. doi:10.1038/oncsis.2015.50 (2016).
23. Stimpson, S E, Coorssen J R, Myers S J. Isolation and identification of ER associated proteins with unique expression changes specific to the V144D SPTLC1 mutations in HSN-I. Biochemistry & Analytical Biochemistry, 5(1), doi:doi:10.4172/2161-1009.1000248 (2015).
24. Dispenzieri, A., et al. Immunoglobulin free light chain ratio is an independent risk factor for progression of smoldering (asymptomatic) multiple myeloma. Blood, 111, 785-789. doi:10.1182/blood-2007-08-108357 (2008).
25. Yamada, K. M., & Araki, M. Tumor suppressor PTEN: modulator of cell signaling, growth, migration and apoptosis. Journal of Cell Science, 114(Pt 13), 2375-2382. (2001).
26. Chang, H., et al. Analysis of PTEN deletions and mutations in multiple myeloma. Leukemia Research, 30(3), 262-265. http://doi.org/10.1016/j.leukres.2005.07.008 (2006).
27. Holman, L., Head, M. L., Lanfear, R., and Jennions, M. D. Evidence of experimental bias in the life sciences: Why we need blind data recording. PLoS Biology, 13. doi:10.1371/journal.pbio.1002190 (2015).
28. Blotta, S., et al. Canonical and noncanonical hedgehog pathway in the pathogenesis of multiple myeloma. Blood, 120(25), 5002-5013. http://doi.org/10.1182/blood-2011-07-368142 (2012).29.
29. Socinki et al. CheckMate 026: A phase 3 trial of nivolumab vs investigator's choice (IC) of platinum-based doublet chemotherapy (PT-DC) as first-line therapy for stage iv/recurrent programmed death ligand 1 (PD-L1)-positive NSCLC. Ann Oncology, 27, Suppl_6:LBA7_PR (2016).
30. Johnson, W. E., Li, C., and Rabinovic, A. Adjusting batch effects in microarray expression data using empirical Bayes methods. Biostatistics (Oxford, England), 8, 118-27. doi:10.1093/biostatistics/kxj037 (2007).
31. Therneau T. A Package for Survival Analysis in S. version 2.38, https://CRAN.R-project.org/package=survival. (2015).
32. Carlson M. hgu133plus2.db: Affymetrix Human Genome U133 Plus 2.0 Array annotation data (chip hgu133plus2). R package version 3.0.0. (2016).
33. Durinck S, Spellman P, Birney E and Huber W. Mapping identifiers for the integration of genomic datasets with the R/Bioconductor package biomaRt. Nature Protocols, 4, pp. 1184-1191. (2009).
34. Schapire, R. E. A brief introduction to boosting. IJCAI International Joint Conference on Artificial Intelligence, 2, 1401-1406. http://doi.org/citeulike-article-id:765005 (1999).
35. Liaw, A. and Wiener, M. Classification and Regression by randomForest. R News 2(3), 18-22. (2002).
36. Meyer, D. et al. e1071: Misc Functions of the Department of Statistics, Probability Theory Group (Formerly: E1071), TU Wien. R package version 1.6-7. http://CRAN.R-project.org/package=e1071 (2015).
37. Van Vliet, M. H. et al., An Assay for Simultaneous Diagnosis of t(4;14), t(11;14), t(14;16)/t(14;20), del1p, add1q, del13q, del17p, MS/MF Expression Clusters, and the SKY-92 High Risk Signature in Multiple Myeloma Patients Haematologica; 98(s1):101. abstract n. P234 (2013).
38. Zhan F, et al. The molecular classification of multiple myeloma. Blood 108(6), 2020-2028. (2006).
39. Kuiper, R., et al. A gene expression signature for high-risk multiple myeloma. Leukemia, 26(11), 2406-2413. http://doi.org/10.1038/leu.2012.127 (2012).
40. Warde-Farley, et al. The GeneMANIA prediction server: Biological network integration for gene prioritization and predicting gene function. Nucleic Acids Research, 38, (SUPPL2) doi:10.1093/nar/gkq537 (2010).

1.-12. (canceled)

13. A machine-implemented method for identifying a gene signature for classifying a patient based upon a likelihood of response to a therapy of interest, the method comprising:
identifying subjects from a first group treated with the therapy of interest that exhibit a greater treatment benefit over a set of genetically similar subjects from a second group of subjects not treated with the therapy of interest as compared to the treatment benefit over a set of random subjects from the second group.

14. The method according to claim 13, wherein genetic similarity is determined based upon expression of functionally coherent gene sets.

15. The method according to claim 14, wherein the functionally coherent gene sets are obtained from a Gene Ontology (GO) category.

16. The method according to claim 14, comprising identifying functionally coherent gene sets that are associated with the genetic similarity to identify the gene signature.

17. The method of claim 13, wherein genetic similarity is determined by defining a classifier Q for each gene set i (Qi) by making a decision boundary defined in terms of an area (distance <γ) around subjects identified from a first group treated with therapy that exhibit a greater treatment benefit over a set of genetically similar subjects from a second group of subjects not treated with therapy as compared to the treatment benefit over a set of random subjects from the second group, such that a hazard ratio for class 1 (all patients that fall into the area) is optimized, wherein class 1 refers to the group of subjects from group 1 expected to respond to the therapy of interest.

18. The method according to claim 17, wherein the decision boundary is such that the hazard ratio is associated with a p-value <0.05.

19. A machine-implemented method for identifying a gene signature for classifying a patient based upon likelihood of response to a therapy of interest from a dataset comprising gene expression data and time until event data for a first group of subjects treated with the therapy and gene expression data and time until event data for a second group of subjects not treated with the therapy, the method comprising:
a) optionally, splitting the subjects into a validation group comprising subjects from both the first and second groups and a training group comprising subjects from both the first and second groups;
b) defining a ranked list of subjects from group 1 that exhibit a greater treatment benefit over a set of genetically similar subjects from group 2 as compared to the treatment benefit over a set of random subjects from group 2,
c) defining a classifier Q for each gene set i (Qi) by making a decision boundary defined in terms of an area (distance <γ) around z top-ranked subjects from step b), wherein z is at least 1, such that a hazard ratio for class 1 (all patients that fall into the area) is optimized, wherein class 1 refers to the group of subjects from group 1 expected to respond to the therapy of interest;
d) determining a performance of classifier Q for each gene set using a gene expression dataset comprising a first group of subjects treated with the therapy and a second group of subjects not treated with the therapy and ranking classifiers Qi based upon their hazard ratios; and
e) selecting top k classifiers as the gene signature for classifying a patient.

20. The method according to claim 19, wherein the treatment benefit is determined for functionally coherent gene sets.

21. The method according to claim 20, wherein the functionally coherent gene sets are obtained from a Gene Ontology (GO) category.

22. The method according to claim 19 wherein K is from 2 to 300.

23. The method according to claim 19, wherein step b) is performed by defining for each subject (i) from the first group of subjects the treatment benefit defined as
$${zPFS}_{i} = \frac{\frac{1}{n} {\sum }_{j ∈ O} ( {PFS}_{i} - {PFS}_{j} ) - μ ( {RPFS}_{i} )}{σ ( {RPFS}_{i} )}$$
wherein O is the set of the n most similar subjects based upon distance from the second group of subjects (i) and (j), PFSi indicates the PFS of subject i and PFSj indicates the PFS of subject j, RPFS indicates a vector of ΔPFSi of patient i with differing random set of patients from the second group in O, μ indicates the mean, and σ indicates a standard deviation.

24. The method according to claim 19, wherein step c) is performed by using a cosine correlation as distance measure.

25. The method according to claim 19, wherein step c) is performed by performing a grid search on all combinations of z and γ.

26. The method according to claim 19, wherein step d) comprises determining the performance of Qi on the validation group of subjects.

27. The method according to claim 19, comprising
f) repeating step a) by splitting the subjects into a new validation group comprising subjects from both the first and second groups and a new training group comprising subjects from both the first and second groups;
g) repeating step b);
h) repeating step c); and
i) determining the performance of classifier Q for each gene set using a gene expression dataset comprising a first group of subjects treated with the therapy and a second group of subjects not treated with the therapy and ranking classifiers Qi based upon mean hazard ratios from step h).

28. The method according to claim 19, wherein the second group of subjects is treated with an alternative therapy.

29. The method according to claim 19, wherein the time until event refers to Progression Free Survival (PFS).
